(19) United States
(12) Patent Application Publication
(10) Pub. No.: US 2025/0367401 A1
(43) Pub. Date: Dec. 4, 2025

(54) METHOD OF FORMING DIFFERENT CONFIGURATIONS FOR THE DISTAL TIP OF CATHETERS, CATHETERS THUS FORMED BY THE METHOD, AND APPARATUS FOR EFFECTING THE METHOD

(71) Applicant: ICU Medical, Inc., San Clemente, CA (US)

(72) Inventor: James Muskatello, Southington, CT (US)

(21) Appl. No.: 19/224,328

(22) Filed: May 30, 2025

Related U.S. Application Data
(60) Provisional application No. 63/654,137, filed on May 31, 2024.

(51) Int. Cl. A61M 25/00

(52) U.S. Cl. CPC A61M 25/001

(57) ABSTRACT

Mold assembly has mold having internal bore and distal section that has at least beveled inner molding surface and forming pin positioned thereat. Space of particular configuration is formed between outer circumferential surface of pin and inner molding surface at distal section of the mold. Space may include beveled inner molding surface and tapered inner molding surface. At least distal section of mold assembly is heated. Catheter having distal portion made of plastic material mounted about mandrel is moved by mandrel into bore so that distal portion of catheter that extends beyond distal end of mandrel is positioned into the space. Heated plastic material at distal portion of catheter flows to fill space to form distal end tip having particular configuration. Particular configuration may be symmetrical or asymmetrical.

### CROSS-REFERENCE TO RELATED APPLICATION

This Patent application claims priority to U.S. Provisional Patent Application No. 63/654,137, filed on May 31, 2024, and entitled “METHOD OF FORMING DIFFERENT CONFIGURATIONS FOR THE DISTAL TIP OF CATHETERS, CATHETERS THUS FORMED BY THE METHOD, AND APPARATUS FOR EFFECTING THE METHOD.” The disclosure of the prior Application is considered part of and is incorporated by reference into this Patent Application.

### FIELD

The present disclosure relates generally to methods and tooling to provide desired tips on catheters for accessing a blood vessel, and more particularly, but not exclusively, to methods and tooling for making intravenous (I.V.) catheters featuring symmetrical or asymmetrical beveled tips.

### BACKGROUND

The present invention relates to methods and tooling to provide a desired tip on catheters including I.V. catheters, and, more particularly, to methods and tooling for forming a beveled tip with a particular configuration on a thin wall catheter constructed of thermoplastic material, for example polyurethane or fluorinated ethylene propylene.

It has long been known that a beveled tip on a catheter for example an I.V. catheter eases the insertion of the catheter into the body of a patient. I.V. catheters and other catheters are variously used for infusing blood, plasma, drugs or other fluids into a patient's body as well as withdrawing blood from the patient. Catheters, when manufactured as original blanks, are cut from tubing stock or extruded. The tubing stock or extruded catheter tubing often can have a relatively blunt ended tip, which can in certain instances be more difficult for a care giver to insert than a catheter with a tapered tip. Therefore, the distal tip of a catheter, for example an I.V. catheter, may be tapered after it is cut to ease its insertion over an introducer needle into the patient.

An example of a method and apparatus for flashless tipping of an I.V. catheter is disclosed in U.S. Pat. No. 4,661,300 (Daugherty). The '300 patent describes a catheter being mounted over a mandrel with the distal end of the mandrel extending beyond the distal end of the catheter. The catheter is carried by the mandrel into a heated pre-configured die so that the distal tip of the catheter is heat formed according to the shape of the symmetrically tapered interior surface of the die. Other methods of tipping an I.V. catheter are disclosed in at least U.S. Pat. No. 5,425,903 (Sloane et al.); U.S. Pat. No. 5,716,572 (Lesiczka et al.); U.S. Pat. No. 5,736,085 (Brown et al.); U.S. Pat. No. 5,795,521 (Mathicu); U.S. Pat. No. 5,843,356 (Bialecki et al.); U.S. Pat. No. 5,985,195 (Muskatello); and U.S. Pat. No. 6,048,485 (Field et al.). Some of the methods of forming a symmetrically tapered frustoconical tip result in catheter plastic material waste, due to the need to trim off excess flash from the distal end of the catheter.

In some medical applications, use of a catheter with a symmetrical frustoconical distal tip may not be desirable as the frustoconical tip may be advanced too far by a care giver into a patient's blood vessel, thereby increasing the risk of infusate infiltration into a wall area of the blood vessel.

Accordingly, there exists a need for a method, and apparatus therefor, of forming both desirable symmetrical and asymmetrical distal end tips for catheters including I.V. catheters.

### SUMMARY

A catheter such as an I.V. catheter is usually made of thermoplastic polyurethane or fluorinated ethylene propylene. In an embodiment, the inventive method mounts a catheter to be tipped, formed or molded onto a mandrel. The mandrel mounted catheter is moved into a bore or passage of a mold that has a cylindrical member such as a cylindrical forming pin fittingly positioned to a distal end region or distal section of the mold. The diameter of the cylindrical forming pin is sized to have approximately the same diameter as, or slightly less than, the inside diameter of the catheter. The passage or bore at the distal section of the mold defines a circumferential inner wall that forms the molding surface of the mold. A space or void is formed or defined between the outer circumferential surface of the forming pin and the inner wall molding surface at the distal section of the mold. The mold and the forming pin fitted thereinto together may be referred to as a mold assembly.

In an embodiment in which a symmetrically tapered catheter tip is to be formed, the mold may have one or more molding surfaces that are symmetrically tapered. In an embodiment in which an asymmetrical catheter tip is to be formed, at least one molding surface of the mold is asymmetrically tapered.

The catheter to be tipped is positioned around, or overlayingly mounted about, a mandrel. The outside diameter of the mandrel is slightly greater than the inside diameter of the catheter, such that the catheter stretches slightly and is friction held onto the mandrel, with the distal end of the catheter extending beyond the distal end of the mandrel. The centerline of the mandrel longitudinally aligns with the centerline of the cylindrical forming pin. The mandrel with the catheter mounted thereabout is moved from the proximal end of the mold into the passage or bore of the mold assembly such that the opening at the distal end of the catheter to be tipped mates with the proximal end of the forming pin and the distal region or portion of the catheter mounts about the proximal end region of the forming pin, with the distal end of the catheter engaging the molding surface at the distal section of the mold assembly. Air contained within the mold assembly may be evacuated out through a through bore of the mandrel as the catheter is about to be tipped. Alternatively, or in combination with air evacuation through the mandrel, air may be evacuated through the forming pin, which may be hollow.

The mandrel does not contact the mold. In embodiments, the mandrel stops short of contacting the forming pin so that the mandrel and forming pin do not come into contact with each other to avoid possible mandrel and/or forming pin wear.

The advance of the mandrel is decelerated near the point at which the catheter initially engages a molding surface of the mold assembly. The distal end region or distal portion of the catheter is then heated in the mold assembly and is slowly moved further into the space in the mold assembly defined between the inner wall at the distal section of the mold and the circumferential outer surface of the forming pin as the distal end of the catheter begins to melt and flow. In an embodiment in which the catheter made of fluorinated ethylene propylene polymer material has a softening temperature of 550° F., the mold may be heated to approximately 575°-650° F. As used herein, flow does not necessarily mean a liquid state, but rather can mean a pliable or softened state of the plastics material due to plastic deformation of the material resulting from being heated.

As the distal end of the catheter is further advanced into the defined space of the mold assembly, the being deformed catheter material flows along and conforms or fills the space or gap with the particular configuration defined between the one or more molding surfaces of the inner wall at the distal section of the mold and the outer wall surface of the cylindrical forming pin. After the distal end tip of the catheter is fully formed, the mold assembly is cooled. Cooling of the mold assembly may be done, for example, with a blast of cooling air or flow of coolant about the distal section of the mold to assist in cooling the catheter tip to solidify it more quickly. The mandrel (with the catheter on it) is then withdrawn from the mold assembly. Finally, the catheter with the formed distal end tip is removed from the mandrel.

It should be appreciated that catheters made from polyurethane or other polymers may also be tipped or molded according to the process disclosed herein with adjustments to mold assembly temperature and/or mold assembly dimensions.

In a symmetrical mold assembly, the plastic material in the distal end region or portion of the catheter flows predominantly longitudinally toward the distal end of the space of the mold assembly predefined by the inner wall surface(s) of the mold and the outer circumferential surface of the forming pin. In an asymmetrical mold assembly, the defined space of the mold assembly may be smaller on one side than on the other. As the distal end region or portion of the catheter is advanced into the defined space of the mold assembly, and as the defined space is being filled by the flowing plastics material, some of the plastics material may flow longitudinally and at an angle toward the larger portion of the defined space.

The mold assembly and the catheter mounted mandrel may be movable relative to each other. In one embodiment, the mold assembly moves toward a stationary mandrel. In another embodiment, the mold assembly is stationary while the mandrel moves towards the mold assembly. In yet another embodiment, the mandrel and the mold assembly move toward each other.

As discussed above, the mold and forming pin are separate components that are fitted together. However, it should be appreciated that the mold and the forming pin may not be separate components, but rather are parts of a single unitary mold assembly.

### BRIEF DESCRIPTION OF THE DRAWINGS

The detailed description of the various embodiments of the invention is with reference to the accompanying drawings, in which:

FIG. 1 is a side view of a mold having symmetrical molding surfaces;

FIG. 2 is a top view of the mold illustrated in FIG. 1;

FIG. 3 is a cutaway side view of the mold having a symmetrical molding surface configuration, along line B-B in FIG. 1;

FIG. 4 is an enlarged detail view of the circled area identified by C in FIG. 3;

FIG. 5 is a cutaway side view of a mold assembly comprising the mold of FIGS. 1-3 with a forming pin in it;

FIG. 6 is a cutaway side view of a catheter mounted on a mandrel inside the passage of the mold assembly of FIG. 5 at the point at which the catheter first contacts the tapered molding surface of the mold;

FIG. 7 is a cutaway side view of a catheter mounted on a mandrel inside the passage of the mold assembly of FIG. 5 and the distal end tip of the catheter having been fully formed;

FIG. 8 is a side view of a catheter having a symmetrical frustoconical distal end tip molded by the mold assembly illustrated in FIG. 5;

FIG. 9 is a side view of a mold for tipping a catheter asymmetrically;

FIG. 10 is a cutaway side view, along line C-C of FIG. 9, of a mold with a configuration having an asymmetric molding surface;

FIG. 11 is an enlarged detail view of the circled area identified by D in FIG. 10;

FIG. 12 is an enlarged detail view of the circled area identified by D in the FIG. 10 mold, after a forming pin has been installed or fitted into the passage having the tapered molding surface at the distal section of the mold to provide an asymmetrical molding assembly;

FIG. 13 is a side view of a catheter having an asymmetrical distal end tip formed by the mold assembly illustrated in FIG. 12;

FIG. 14 is a side view of another asymmetrically tipped catheter formed by a mold assembly similar to the mold assembly of FIG. 12, but with its beveled molding surface extending around the inner wall of the mold;

FIG. 15 is a side view of yet another catheter having an asymmetrical distal end tip formed by a mold assembly that has only one beveled molding surface at the inner wall of the mold, and

FIG. 16 is a flow chart illustrating the steps of an exemplar method of the present invention.

### DETAILED DESCRIPTION

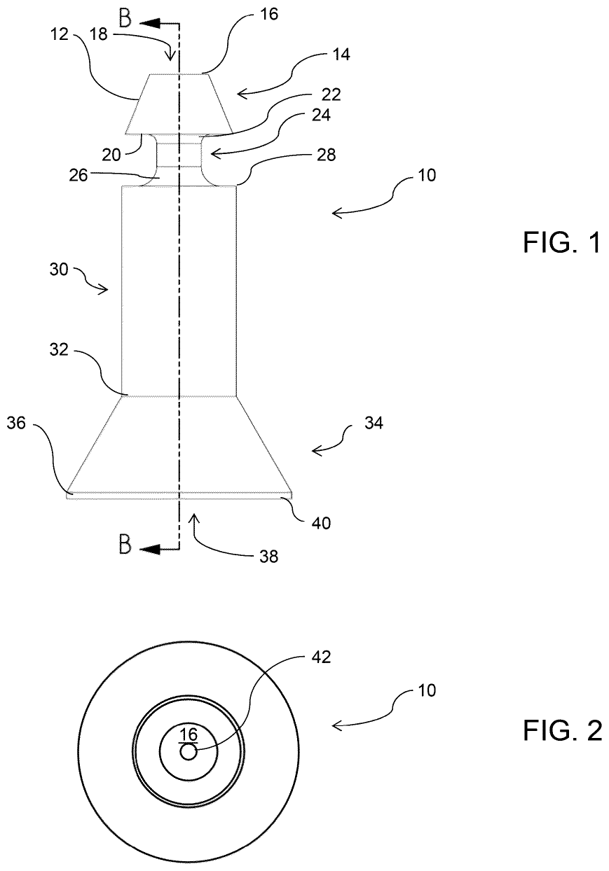

With reference to the mold 10 shown in FIG. 1, it should be appreciated that the term “distal” discussed herein refers to the direction pointing towards the reference number 18 of mold 10, whereas the term “proximal” refers to the direction pointing towards reference number 38 of mold 10.

Referring to FIGS. 1-4, a mold 10 has an upper conical frustum portion 12 in a distal end region or distal section 14, a conical frustum lower proximal section 34 and a cylindrical portion or section 30 sandwiched by the distal section 14 and the proximal section 34. The upper conical frustum portion or distal section 12 has a top surface 16 at the distal end 18 of mold 10 and a bottom surface 20. A narrowed cylindrical neck portion 24 integrally joins the distal section 12 to the cylindrical portion 30 via an upper radius 22 at the lower surface 20 of distal section 12 and a lower radius 26 at upper surface 28 of cylindrical portion 30, respectively. Neck portion 24 serves to limit heat transfer from the distal section 12 to the rest of mold 10 as will be discussed infra. The cylindrical portion 30 adjoins the proximal section 34 at junction 32. Proximal section 34 has a proximal end 36 that forms a base 40 of the mold 10.

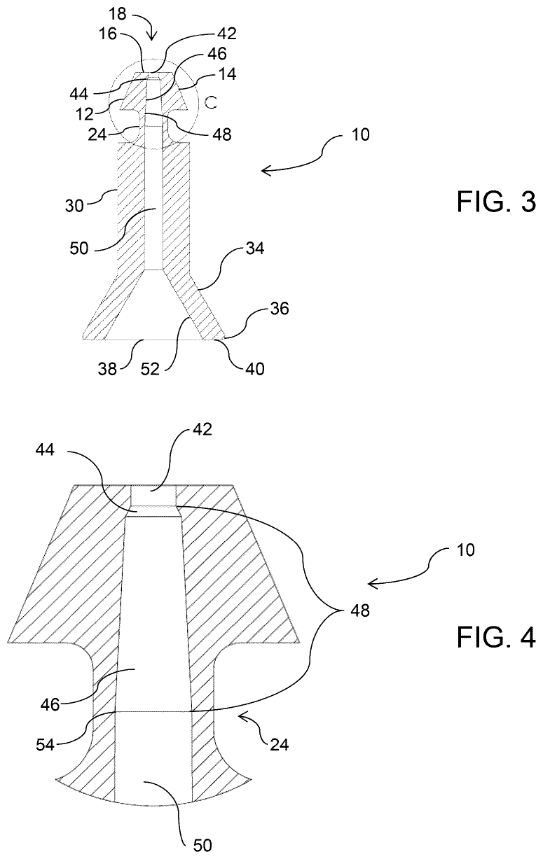

As shown in FIGS. 3-4, a bore or through passage 50 extends longitudinally along the central axis of mold 10 from junction 32 through cylindrical section 30 and substantially through distal section 14. A hole or aperture 42 connects the bore 50 via a particularly configured primary bevel surface (or simply “beveled molding surface”) 44 to the top surface 16 of mold 10. Bore 50 may also be referred to as a passage of the mold 10. The beveled molding surface 44 may also be referred to as the molding surface of a particular configuration that is provided by the mold assembly at the inner wall of the distal section 14 of the mold 10. In embodiments, the angle of beveled molding surface 44 relative to the longitudinal axis of the mold may be, for example, in the range of 15 to 35 degrees.

Further with reference to FIGS. 3-4, aperture 42 extends from distal end 18 to the beveled molding surface 44 in the upper conical frustum portion or distal section 12. Beveled molding surface 44 has a frustoconical shape and merges into a secondary bevel molding surface (or “tapered molding surface”) 46 in distal section 14. In embodiments, the angle of the tapered molding surface 46 relative to the longitudinal axis of the mold may be, for example, in the range of 1 to 5 degrees. In combination, beveled molding surface 44 and tapered molding surface 46 may form a particular configuration 48 of the inner wall molding surface of the distal section 14 of the mold 10. It should be appreciated that only the beveled inner molding surface 44 may be needed for forming a distal end tip of a particular configuration for a catheter. The tapered molding surface 46 extends to the distal end of bore 50, which is represented by transition 54 shown in FIG. 4.

In an embodiment, the mold may be configured to have only one of primary bevel or beveled molding surface 44 and secondary bevel or tapered molding surface 46. In this embodiment, the beveled molding surface present provides the molding surface configuration.

As shown in FIG. 4, transition 54 is where the tapered molding surface and the inner wall of the bore 50 is joined, and where the plastics material at the distal portion of the cannula catheter, when heated, may flow into the space defined between the inner molding surface of the mold and the outer surface of the forming pin such that the distal portion, and the distal end tip, of the catheter may be molded into a particular configuration.

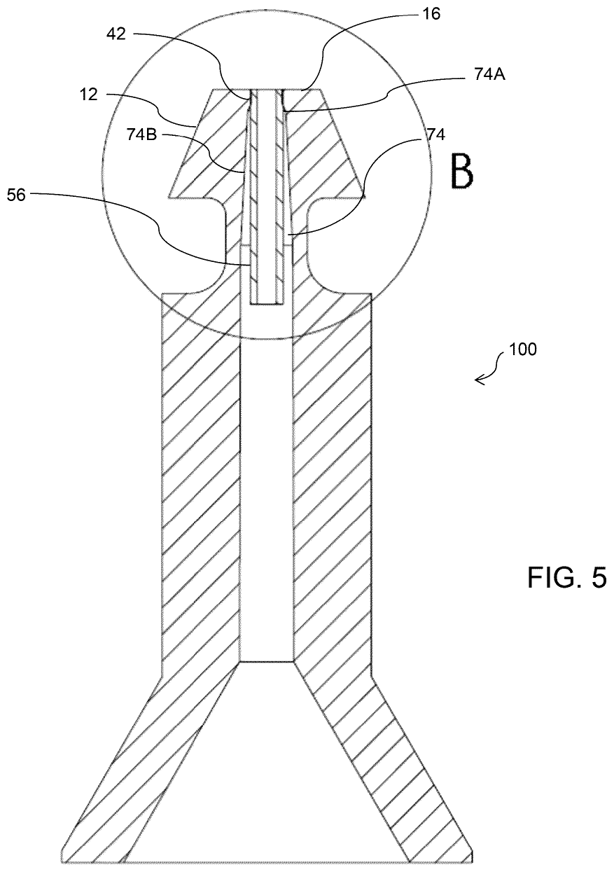

FIG. 5 illustrates an exemplar mold assembly 100 of the present invention. As shown, mold 10 is assembled by inserting cylindrical forming pin 56 through aperture 42. It should be appreciated that cylindrical forming pin 56 may be completely or partially fitted inside the mold. In the FIG. 5 illustration, forming pin 56 is shown to be completely inserted into mold 10 such that the top of forming pin is flush with the distal end 18, or top surface 16, of mold 10. Cylindrical forming pin 56 may be hollow, as illustrated, or solid. In some embodiments, cylindrical forming pin 56 is press fit into aperture 42 to securely hold it longitudinally along bore 50 relative to the molding surfaces 44 and 48 of mold 10. In some embodiments, a portion of the cylindrical forming pin 56 may extend distally above top surface 16 and may optionally be mounted to the upper conical frustum portion 12 by fasteners or by welding. As shown, a space 74 is defined between the outer circumferential surface of forming pin 56 and the inner wall molding surfaces 44 and 48 at the distal section of mold 10. For the FIG. 5 embodiment, the portion of space 74, designated 74A, formed between beveled molding surface 44 and forming pin 56 has the same beveled dimension as it circumscribes about forming pin 56. The portion of space 74, designated 74B, formed between the outer wall of forming pin 56 and the tapered inner wall molding surface 48 tapers outwardly from its distal end that connects to beveled molding surface 44 to its proximal end at transition 54 that connects to the bore 50 of the cylindrical portion 30.

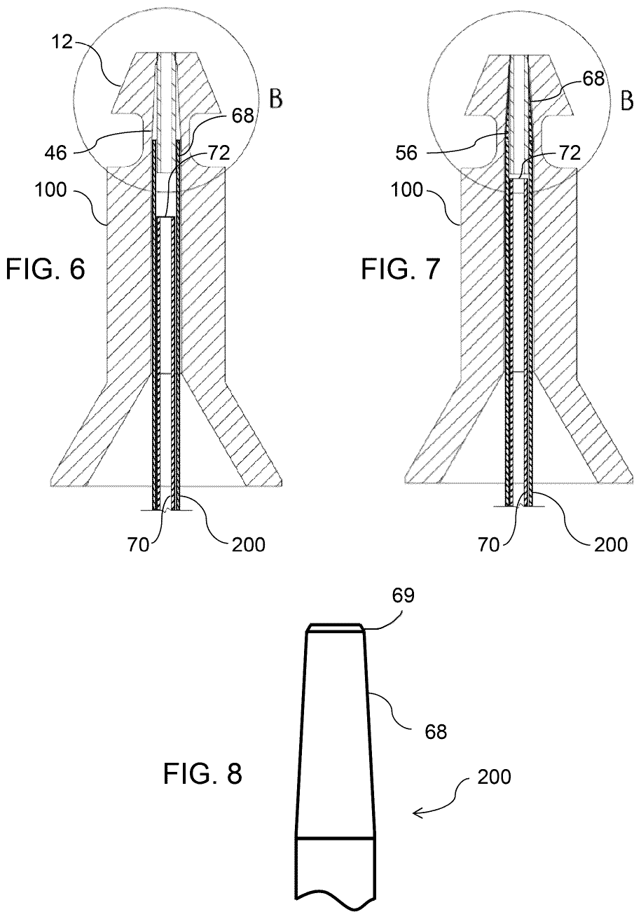

The operation of the method of the present invention may be gleaned from FIGS. 6-7. FIG. 6 shows the catheter 200 mounted about mandrel 70 having been moved along bore 50 into mold assembly 100, and the distal end portion 68 of catheter 200 is in contact with the beveled molding surface 44 and the tapered molding surface 46. More specifically, the proximal portion of the forming pin 56 is fitted into a section of the lumen at the distal portion of the catheter 200 defined by the inner wall of the catheter while the outer wall of the catheter is in contact with the molding surfaces 44 and 46. As the distal section 12 of mold 100 is heated as discussed above, the distal end portion 68 of catheter 200 begins to melt. In an embodiment, the heating of the distal section 12 of the mold may be by RF (radio-frequency) or other known wireless and direct in contact heating means. The neck portion 24, since having a smaller diameter than the rest of the mold, serves to limit heat transfer from the distal section 14 of the mold to its cylindrical portion 30. Given that the molding of the distal end portion of the catheter is confined in a predefined space of the desired configuration, unlike the forming of the distal end region of a catheter such as for example described in the above-noted references, there is no waste of the plastics material in the present invention as the distal end portion of the catheter is formed in a predefined space with the particular desired configuration, such that there is no need to remove excess flash at the distal end of the catheter as in the prior art. So, too, by not having to take the extra step of removing the excess flash, the present invention may also save the processing time for forming the distal end portions of catheters.

FIG. 7 shows that, as distal end portion 68 of catheter 200 is heated and its plastics material flows, mandrel 70 is advanced distally along bore 50 to move the distal end portion 68 of the catheter further along distal section 12 of mold assembly 100 such that the plastic material at the distal end of catheter 200 is meltingly molded into a distal end tip having the particular configuration defined between the inner wall molding surface 44 and 48 of the mold and the outer circumferential surface of the forming pin 56. As shown, the distal end 72 of mandrel 70 stops short of contacting the proximal end of cylindrical forming pin 56. Thus, the distal portion of the catheter 200 distal of the distal end 72 of mandrel 70 is heat molded to have the particular configuration defined by the beveled molding surface 44 and the tapered molding surface 46 of mold 100, in conjunction with the outer circumferential surface of forming pin 56. When mold assembly 100 is cooled, the catheter 200 now formed with a particular configuration at its distal end portion may be withdrawn from mold assembly by proximal movement of mandrel 70 relative to mold assembly 100. Catheter 200 is subsequently removed from mandrel 70.

FIG. 8 shows a catheter 200 with its distal end portion 68 tipped or molded with a symmetrical configuration by mold assembly 100.

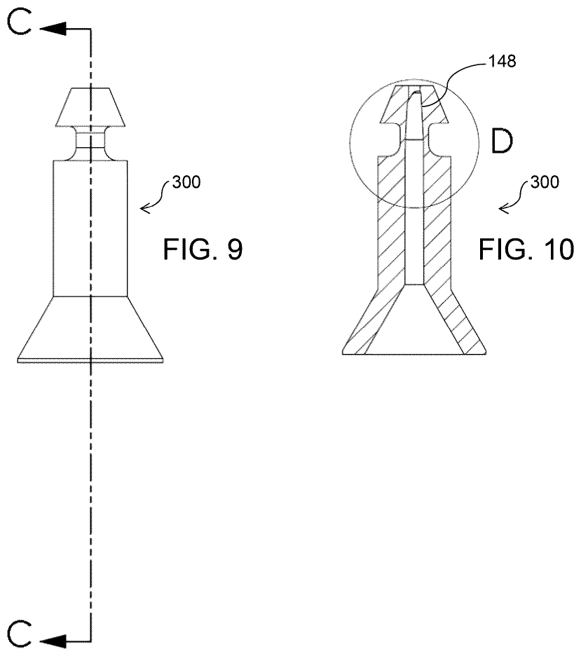

FIG. 9 shows a mold 300 for asymmetrical tipping or molding of a catheter tip. Although externally it may look like the symmetrical mold 100 discussed above, mold 300 is a mold that has asymmetrical mold forming surface(s) for asymmetrically forming the distal end portion of a catheter.

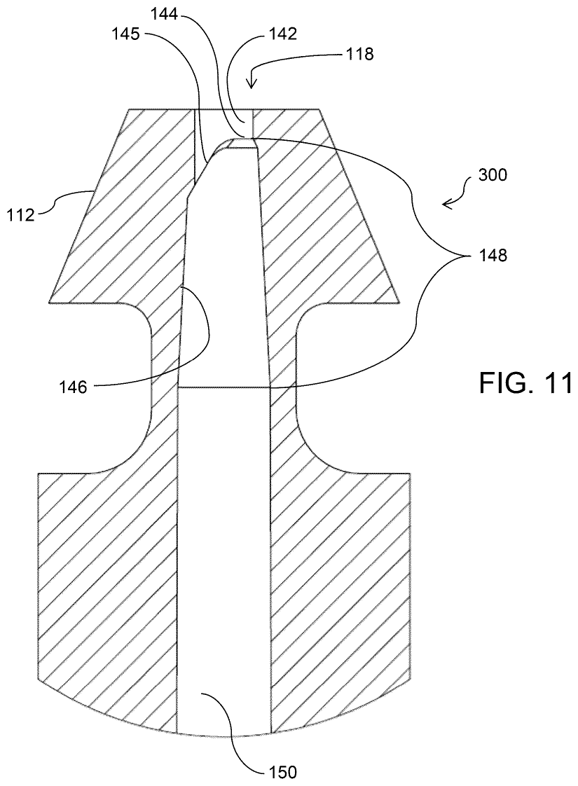

The asymmetrical molding feature of mold 300 is shown in FIGS. 10-11. FIG. 11 shows the enlarged view of the portion D of the mold shown in FIG. 10. As shown, an aperture 142 opens at distal end 118 and extends proximally to the beveled molding surface 144 inside distal section 112. Beveled molding surface 144 may be of different non-symmetrical configurations and be referred to as asymmetrical configurations. For the exemplar asymmetrical configuration of the FIG. 11 embodiment, beveled molding surface 144 extends proximally along one of its sides to meet with the distal end of the tapered molding surface 146. Along another side opposite to the one side, beveled molding surface 144 extends laterally to an angled molding surface 145, which in turn extends to the tapered molding surface 146. The beveled molding surface 144, the angled molding surface 145, and the tapered molding surface 146 cooperate to form the particular molding surface configuration designated by reference number 148. The tapered molding surface 146 extends proximally and communicatively connects to the central bore 150 of mold 300.

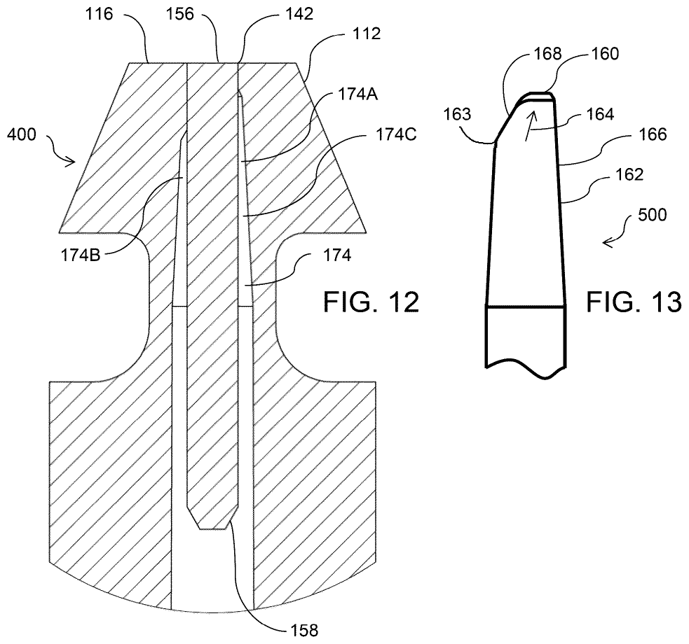

FIG. 12 is an illustration of another mold assembly 400 that has a cylindrical forming pin 156 inserted through aperture 142 to define the space 174 between the forming pin and the beveled and tapered molding surfaces whereby the distal end region or portion of a catheter may be molded or tipped into a particular configuration. Looking at the cross-sectional view of the FIG. 12 embodiment in the direction into the paper, space 174 has at its tapered space portion 174C more space 174A on one side and less space 174B on the other side. The tapered space portion tapers outwardly, i.e., being wider, toward the proximal end of the mold assembly. Similar to the previous mold assembly embodiments, cylindrical forming pin may be completely, or partially, fitted inside the mold. For the embodiment of FIG. 12, the proximal end 158 of cylindrical forming pin 156 is chamfered to assist in guidingly mounting the distal end portion of the catheter onto the proximal end portion of the forming pin. In other embodiments, a portion of the cylindrical forming pin 156 may extend above the top surface 116 of the mold and may optionally be mounted to the upper conical frustum portion or distal section 112 by fasteners or by welding.

FIG. 13 shows a catheter 500 that was molded or tipped in a mold assembly such as the mold assembly 400 of FIG. 11. The asymmetrical configuration of catheter 500 has a beveled distal end tip 160 that extends to an angled surface 168 and a longitudinal circumferential tapered surface 162. In the asymmetrical tipping or molding of the distal end portion of catheter 500, arrow 164 indicates that the plastics material in the distal end portion 166 of catheter 500 is confined to flow both longitudinally and circumferentially in the space defined by the inner wall molding surfaces of the mold and the outer circumferential surface of the forming pin of mold assembly 400.

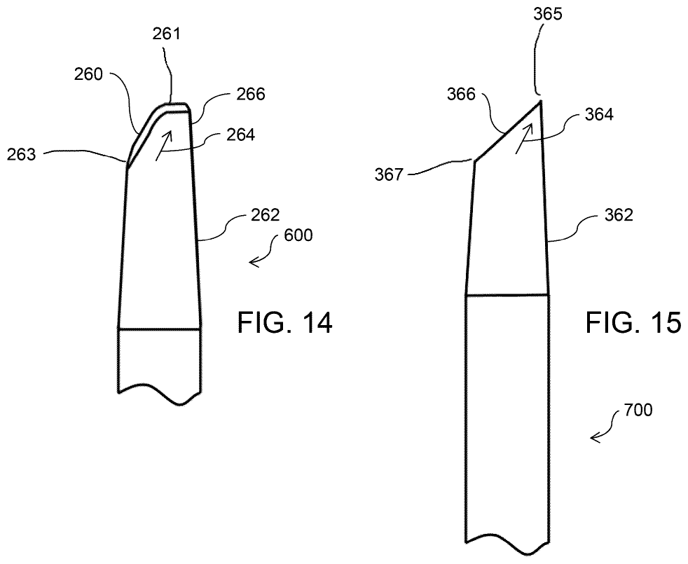

FIG. 14 shows another catheter 600 that has an asymmetrical configured distal end, but with the beveled end 260 circumscribing around the catheter. Catheter 600 also is shown to have a longitudinal and circumferential tapered surface 262 extending proximally from beveled end 260. Arrow 264 indicates that the plastics material in the distal end portion 266 of catheter 600, when being heatedly molded, flows at an angle both longitudinally and circumferentially in pre-defined space at the distal section of the mold assembly.

FIG. 15 shows a catheter 700 tipped or molded to have an asymmetrical configuration. For the FIG. 15 catheter, there is only a single beveled surface 362. As indicated by arrow 364, when being formed, the plastics material at the distal end portion 366 of catheter 700 flows at an angle both longitudinally and circumferentially in the pre-defined space at the distal section of the mold assembly.

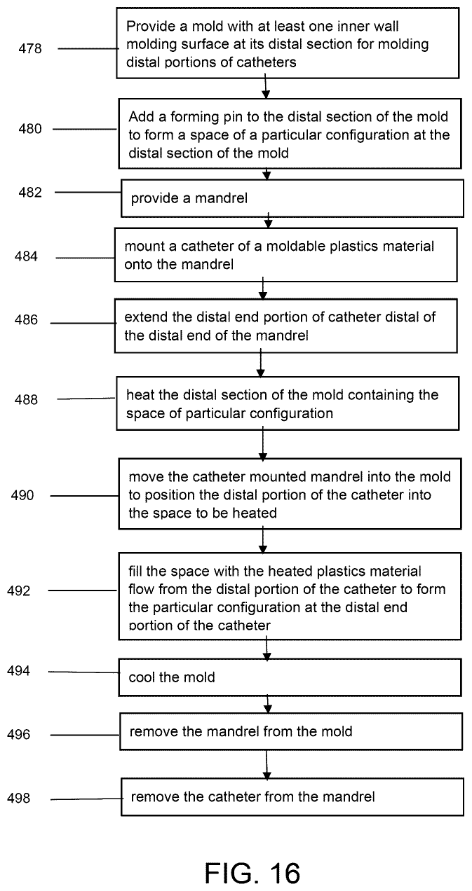

FIG. 16 is a flow chart 800 that shows the steps of an exemplar method 800. Starting at step 478, a mold having an internal bore and a distal section including a particularly configured inner molding surface is provided. At step 480, a mold assembly is effected by the positioning of a forming pin into the distal section of the mold such that a space of a particular configuration is formed between the outer surface of the forming pin and the inner molding surface of the distal section. At step 482, a mandrel movable relative with the mold is provided. At step 484, a catheter having a distal portion with a distal end and made from a plastic material is mounted about the mandrel. At step 486, the distal portion of the catheter is extended distal or beyond the distal end of the mandrel. At step 488, at least the distal section of the mold assembly that contains the space is heated. At step 490, the mandrel having the catheter mounted thereabout is inserted into a bore of the mold so that the mandrel is moved into the mold and the mold and the distal portion of the catheter is moved into the predefined space of the mold to be heated. At step 492, the heated plastics material at distal portion of the catheter flows in to fill the space to form the particular configuration at the distal end portion of the catheter. At step 494, the mold is cooled. At step 496, the mandrel with the catheter mounted thereabout is removed from the mold assembly. At step 498, the catheter with the particular configuration at its distal portion is removed from the mandrel and the process ends.

It should be appreciated that the descriptions of the subject matter above are illustrative only and not limiting. Features described in one embodiment may be combined with features describing in other embodiments. Accordingly, it is intended that the invention be limited only by the spirit and scope of the hereto appended claims.

What is claimed is:

1. For a catheter having a distal end tip, a method of configuring the distal end tip of the catheter, comprising:
providing a mold having an internal bore and a distal section including a particularly configured inner molding surface,
positioning a forming pin within the distal section of the mold such that a space of a particular configuration is formed between the outer surface of the forming pin and the inner molding surface of the distal section, the mold and the forming pin together defining a mold assembly;
mounting a catheter having a distal portion with a distal end and made from a plastic material about a mandrel having a distal end, the distal end of the catheter extending beyond the distal end of the mandrel;
heating at least the distal section of the mold assembly that contains the space;
inserting the mandrel having the catheter mounted thereabout into the bore of the mold such that the distal portion of the catheter is moved into the space to be heatedly molded to form a catheter having a distal end tip of the particular configuration.

2. The method of claim 1, further comprising;
cooling the mold assembly;
removing the mandrel with the catheter mounted thereabout from the mold assembly; and
removing the catheter with the particular configuration at its distal end tip from the mandrel.

3. The method of claim 1, wherein the particular configuration of the space formed between the inner molding surface and the outer surface of the forming pin is symmetrical.

4. The method of claim 1, wherein the particular configuration of the space formed between the inner molding surface and the outer surface of the forming pin is asymmetrical.

5. The method of claim 1, wherein the mandrel has a hollow interior.

6. The method of claim 5, further comprising evacuating air from the mold assembly through the interior of the mandrel during insertion of the catheter.

7. The method of claim 5, further comprising blowing air into the mold assembly through the interior of the mandrel during withdrawal of the catheter.

8. The method of claim 1, wherein the forming pin is hollow.

9. The method of claim 8, further comprising evacuating air from the mold assembly through the interior of the forming pin during insertion of the catheter.

10. The method of claim 8, further comprising blowing air into the mold assembly through the interior of the shape forming pin during withdrawal of the catheter.

11. A catheter, comprising:
an elongated tubular body made of plastics material moldable by heat having a distal end portion that has an asymmetrical tip of a particular configuration formed by the plastics material heated to flow at an angle longitudinally and circumferentially into an asymmetrical space within a mold during tipping of the catheter, the asymmetrical space defined between at least one inner molding surface of the mold having the particular configuration and a forming pin fitted into at least a section at the distal end portion of the catheter when the catheter is being tipped.

12. A mold assembly for forming a distal end portion of a catheter into a particular configuration, comprising:
a mold having a bore extending through a cylindrical section to a distal section, the distal section having an inner wall surface, at least one part of the inner wall having a particular configuration, an aperture formed at the distal end of the distal section that extends into the bore;
a forming pin fitted through the aperture into the space defined by the inner wall surface of the distal section, the forming pin having an outer circumferential surface that does not contact the inner wall surface such that a space of a particular configuration is formed between the outer surface of the forming pin and the inner wall of the tapered distal section;
wherein at least the distal section of the mold containing the space of the particular configuration is heated such that when a distal portion of a catheter extending beyond a distal end of a mandrel onto which the catheter is mounted is moved into the space, the distal portion is heatedly molded to form a distal end tip having the particular configuration of the space.

13. The mold assembly of claim 12, wherein the inner wall of the distal section includes a beveled inner molding surface adjacent to the distal end of the distal section and a tapered inner molding surface extending proximally from the beveled inner molding surface such that the space of the particular configuration has a distal beveled inner molding surface and a proximal tapered inner molding surface;
wherein the distal portion of the catheter formed in the space has a distal end tip that has a beveled configuration of the particular configuration and a tapered portion proximal of the distal end tip.

14. The mold assembly of claim 12, wherein the beveled inner molding surface at the distal section of the mold is symmetrical such that the distal end tip of the catheter molded in the mold assembly has a symmetrical configuration.

15. The mold assembly of claim 12, wherein the beveled inner molding surface at the distal section of the mold is symmetrical such that the distal end tip of the catheter molded in the mold assembly has a symmetrical configuration.

16. The mold assembly of claim 12, wherein the beveled inner molding surface at the distal section of the mold is asymmetrical such that the distal end tip of the catheter molded in the mold assembly has an asymmetrical configuration.
